(19) United States
(12) Patent Application Publication
(10) Pub. No.: US 2024/0161880 A1
(43) Pub. Date: May 16, 2024

(54) Method for Tracking and Managing Specimens Collected at Remote Collection Locations

(71) Applicant: Lifescan Labs of Illinois, LLC, Skokie, IL (US)

(72) Inventor: Eliyahu Kutoff, Skokie, IL (US)

(73) Assignee: Lifescan Labs of Illinois, LLC, Skokie, IL (US)

(21) Appl. No.: 18/142,884

(22) Filed: May 3, 2023

Related U.S. Application Data
(60) Provisional application No. 63/425,388, filed on Nov. 15, 2022.

(51) Int. Cl. G16H 10/40; G16H 10/60; G16H 40/63; G16H 40/67

(52) U.S. Cl. CPC G16H 10/40; G16H 10/60; G16H 40/63; G16H 40/67

(57) ABSTRACT

A method for tracking medical specimens includes, at a first location, preparing a specimen container comprising an identification label having a unique, machine-readable code. Test orders are received and recorded on an electronic test order database. The test orders specify one or more specimens to be collected from a patient at a remote patient specimen collection location. The specimen container is transported to the remote patient specimen collection location and the one or more specimens are collected into the specimen container. The identification label's unique, machine-readable code is scanned, and the unique, machine-readable code is recorded on the electronic test order database for electronically associating the specimen container with the patient.

### REFERENCE TO RELATED APPLICATIONS

This application claims priority under 35 U.S.C. 119(e) to U.S. provisional application Ser. No. 63/425,388 filed on Nov. 15, 2022, entitled Methods for Tracking and Managing Specimens Collected at Remote Collection Locations, the disclosure of which is hereby incorporated herein by reference.

### BACKGROUND OF THE INVENTION

### 1. Field of the Invention

The present invention relates to the tracking and management of medical specimens. More particularly, the present invention relates to methods of tracking and managing the collection and transportation of specimens collected at remote specimen collection locations by mobile medical technicians, and the flow of the patient and specimen data related thereto.

### 2. Background

A variety of medical specimens are used for the diagnosis and treatment of patients. In many cases, it is desirable for the medical specimens to be collected from the patient at the patient's residence or at a designated collection facility. After the medical specimens are collected, the specimen must be transported to a separate testing facility where a variety of diagnostic tests can be performed.

The results from testing of medical specimens have significant implications for patients. Medical specimens that are lost, improperly identified, drawn in incorrect or expired test tubes, and/or processed in an untimely manner can lead to severe adverse health consequences for the patients and can cause serious processing and payment issues for the labs and technicians collecting the specimens. As such, it is imperative that the medical specimens are properly identified and tracked, and a chain of custody is maintained, to ensure that the specimens are correctly identified and that the test results are accurately reported.

Traditionally, once a medical specimen is collected, the specimen is associated with the patient using information such as, for example, the address the specimen was collected at; the name, date of birth, and/or other identifying information of the patient; the type of specimen collected; and the type of testing to be performed on the specimen. Some of this information is typically handwritten on a label which has been applied to the specimen container. Alternatively, this information can be applied to the specimen container by printing and securing, via a mobile label printer, a barcode or other identification label to the specimen container following collection of the sample. The specimen is then transported to a testing facility for processing.

Prior methods of tracking and managing transportation of medical specimens leave considerable room for error or misidentification when the wrong information is handwritten on the sample, the technician identifies the wrong patient, the wrong test is assigned to the specimen, the specimen is drawn in an incorrect or expired test tube, the test tube is not properly filled, or the technician's handwriting is illegible. Additionally, current mobile systems for printing barcodes and/or identification labels are often unreliable and may require significant servicing and troubleshooting to operate properly.

There are known methods of tracking and managing transportation of medical specimens which incorporate prelabeled specimen containers that associate a particular specimen to a patient, for example, by providing a peelable barcode label which can be applied to associated testing paperwork. Examples of prior methods incorporating these peelable barcode labels are shown and described in Stevens et al. U.S. Pat. No. 6,599,451 and Stevens et al. U.S. Pat. No. 7,122,157. There are also known methods of tracking and managing transportation of medical specimens which incorporate prelabeled specimen containers that can be associated with a barcode printed on an identification belt/bracelet worn by a patient. Examples of prior methods incorporating these prelabeled specimen containers are shown and described in Kitao U.S. Pat. No. 9,633,177.

There are also known methods of tracking and managing transportation of medical specimens which incorporate specimen containers having RFID tags or other electronic identification elements that can be associated with a particular specimen, patient, and test using an electronic device. Examples of prior methods incorporating RFID tags or other electronic identification elements are shown and described in Taylor U.S. Pub. No. 2011/0047092; Bolander et al. U.S. Pub. No. 2012/0025985; Egbert U.S. Pub. No. 2010/0315205; and Abrams et al. U.S. Pat. No. 7,070,053.

However, the methods incorporating peelable labels require manual entry of the patient's identification information which is subject to the same errors and misidentifications as the traditional method of tracking and managing transportation. Additionally, the RFID tagged containers and the specimen containers incorporating other electronic identification elements are costly and time consuming to produce.

Accordingly, there exists a need for an improved, more accurate, cost-effective, and time efficient method for tracking and managing medical specimens.

### SUMMARY OF THE INVENTION

In the present invention, a method for tracking specimens comprises, at a first location, preparing a specimen container having an identification label. The identification label can include a unique, machine-readable code. A test order can be received and recorded on a test order database. The test order can identify a patient and can specify one or more specimens to be collected from the patient at a remote patient specimen collection location. The specimen containers can be transported to the remote patient specimen collection location and the one or more specimens can be collected into the specimen container. The identification label's unique, machine-readable code can be scanned, and the machine-readable code can be recorded to the test order database for electronically associating the specimen container with the test order and the patient.

Preferably, the specimen container includes a cap member having a color code. Preferably, the machine-readable code is an alphanumeric code. Yet more preferably, the machine-readable code is an alphanumeric code, wherein the alphanumeric machine-readable code includes a first code portion that is electronically associated with the color code of the cap member.

Preferably, the test order specifies one or more patient identifiers. Preferably, the patient can be identified by directing the patient to recite from memory a recited patient identifier and comparing the recited patient identifier with the one or more patient identifiers specified on the test order. Yet more preferably, the recited patient identifier can be recorded on the electronic test order database, and the recited patient identifier can be compared with the one or more test order patient identifiers.

Preferably, the machine-readable code can be a visual, machine-readable code. Yet more preferably, the machine-readable code is a barcode or a QR code.

Preferably, the test order specifies a first color code of the cap member. Yet more preferably, the first code portion of the alphanumeric barcode can be compared with the first color code specified on the test order, wherein, if the first code portion is not electronically associated with the first color code, the specimen is rejected.

Preferably, the specimen container can be transported to a third location and one or more tests can be performed on the one or more specimens. Yet more preferably, the specimen container can be transported to a testing facility and one or more tests can be performed on the one or more specimens. Preferably, one or more results of the one or more tests can be recorded on the electronic test order database.

Preferably, the identification label's machine-readable code can be scanned, and the unique, machine-readable code can be transmitted to the electronic database, using a mobile device comprising a code scanning device, an input device, a display device, and a processor. The code scanning device, the input device, and the display device can be in communication with the processor, and the mobile device can be in wireless communication with the electronic database.

In another embodiment of the present invention, a method for collecting and storing in an electronic database information about medical specimens comprises, at a first location, preparing a specimen container having an identification label including a unique, machine-readable code. A test order can be received, the test order specifying a patient, one or more patient identifiers, one or more specimens to be collected, and a remote patient specimen collection location. The test order can be recorded on an electronic test order database. The specimen container can be transported to the remote patient specimen collection locations, and the specimen can be collected into the specimen container. The identification label's unique, machine-readable code on the specimen container can be scanned, and the unique, machine-readable code can be transmitted to the electronic database for thereby electronically associating the specimen container with the specimen and the patient.

Preferably, the identification label's machine-readable code can be scanned and transmitted using a mobile device comprising a code scanning device, an input device, a display device, and a processor. The code scanning device, the input device, and the display device are in communication with the processor, and the mobile device is in wireless communication with the electronic database.

### BRIEF DESCRIPTION OF THE DRAWINGS

The above-mentioned and other features of this invention and the manner of attaining them will become more apparent, and the invention itself will be better understood by reference to the following description of the embodiments of the invention, taken in conjunction with the accompanying drawings, wherein:

FIG. 1 is a block diagram illustrating an electronic test order information processing system and a data flow for tracking and managing collection and transportation of specimens by mobile technicians in accordance with the principles of the present invention;

FIGS. 2-8 are flowcharts illustrating a method of tracking and managing collection and transportation of specimens by mobile technicians in accordance with the principles of the present invention;

FIGS. 9-10 are block diagrams illustrating the method of tracking and managing collection and transportation of specimens detailed in FIGS. 2-8; and,

FIG. 11 is a block diagram illustrating the method of tracking and managing collection and transportation of specimens detailed in FIG. 2.

Corresponding reference characters indicate corresponding parts throughout several views. Although the exemplification set out herein illustrates certain embodiments of the invention, the embodiments disclosed below are not intended to be exhaustive or to be construed as limiting the scope of the invention to the precise form disclosed.

### DETAILED DESCRIPTION OF THE PREFERRED EMBODIMENTS

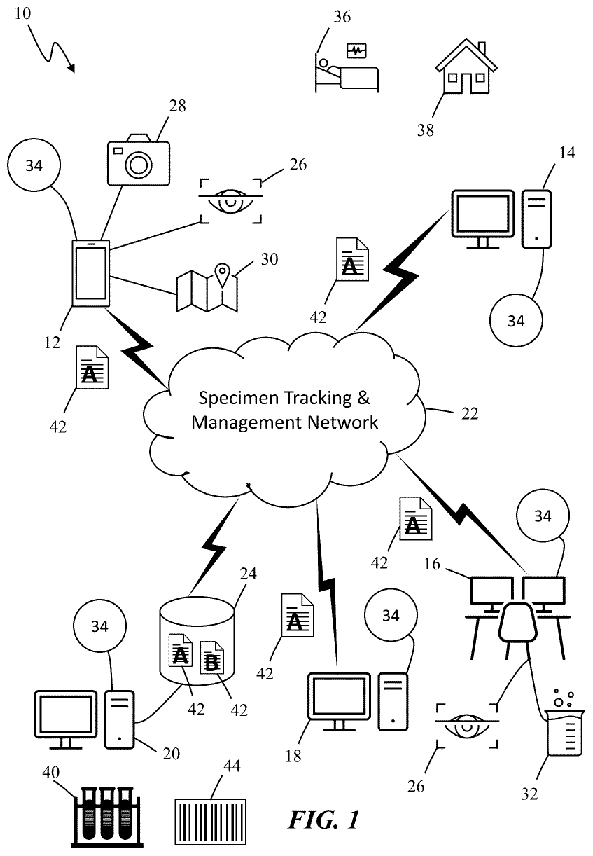

FIG. 1 illustrates an electronic test order processing system 10 which can be adapted for tracking and managing the collection and transportation of specimens by mobile technicians, and the flow of the patient and specimen data related thereto, in accordance with the principles of the present invention. The test order system 10 includes, but is not limited to, one or more mobile devices 12, one or more specimen ordering devices 14, one or more testing instruments 16, one or more dispatch center devices 18, and one or more test order servers 20.

The mobile devices 12, ordering devices 14, testing instruments 16, dispatch center devices 18, and test order servers 20 are in communication with a specimen tracking/management network 22 via one or more wired and/or wireless communication interfaces. The network 22 can be adapted such that electronic data can be freely transmitted between, and received by, the devices connected to the network 22.

The test order servers 20 can include one or more processors and one or more non-transitory machine-readable mediums. The non-transitory machine-readable mediums can include one or more test order databases 24 adapted to store and catalog electronic data. The test order servers 20 can include, but are not limited to, file servers, desktop and laptop computers, and other types of electronic information servers.

The mobile devices 12 can include, but are not limited to, mobile phones, tablet computers, smart phones, personal digital assistants, laptop computers, and other types of moveable processing devices. For example, the mobile devices 12 can be smart phones such as the iPhone by Apple, Inc., android based smart phones, and/or smart phones operating on other operating systems.

The mobile devices 12 can include code sensors 26 adapted to read and record visual, machine-readable code; cameras 28 adapted to capture both still photographs and videos; and location tracking devices 30. The location tracking devices 30 can include, for example, global positioning system (GPS) transmitters/receivers. Each mobile technician is provided with a mobile device 12 such that mobile technicians can receive electronic data from, and transmit electronic data to, the other devices connected to the network 22.

The ordering devices 14, the testing instruments 16, and the dispatch center devices 18 can include, but are not limited to, multimedia capable desktop and laptop computers, tablet computers, mobile phones, smart phones, personal digital assistants, and other types of network devices. Additionally, the testing instruments 16 can include code sensors 26 adapted to read and record visual, machine-readable code, and diagnostic machines 32 adapted to perform various diagnostic tests on the specimens 40.

The ordering devices 14 can create and transmit, via the network 22, a desired test order 42 requesting the collection of one or more specimens 40 from a patient 36, the specimens 40 to be collected at a remote patient specimen collection address 38 listed on the test order 42. The test order 42 can be created in a specimen ordering/tracking program 34 installed on the ordering devices 14. After a test order 42 has been created, the test order 42 is transmitted to the dispatch center devices 18 whereby dispatch personnel can review the test order 42 and thereafter transmit the test order 42 to the test order servers 20 for recording on the database 24.

In addition to being installed on the ordering device 14, the specimen ordering/tracking program 34 can be installed on each of the devices connected to the network 22 including, for example, the mobile devices 12, testing instruments 16, dispatch center devices 18, and test order servers 20 for thereby facilitating the transmission of the test orders 42 between the devices, via the network 22, and providing a uniform, easily accessible user interface for viewing the test orders 42 and tracking and managing the collection and transportation of the specimens 40.

The specimen tracking/management network 22 can include, but is not limited to, a wired and/or wireless communications network comprising one or more portions of: the internet, an intranet, a local area network, a wireless local area network, a wide area network, a metropolitan area network, a public switched telephone network, a wireless personal area network, a cloud server network, and other types of wired and/or wireless communications networks.

The network 22 can comprise one or more gateways, routers, bridges and/or switches. A gateway connects two or more computer networks that use different network protocols and/or operate at different transmission capacities. A router receives transmitted messages and forwards them to their correct destinations over the most efficient available route. A bridge is a device that connects two or more networks that use the same communications protocols so that information can be passed from one network device to another. A switch is a device that filters and forwards data packets between network segments based on a pre-determined sequence (e.g., timing, sequence number, etc.).

An operating environment for the devices connected to the specimen tracking/management network 22 can include one or more central processing units, processors, memories, and/or other types of non-transitory machine-readable mediums.

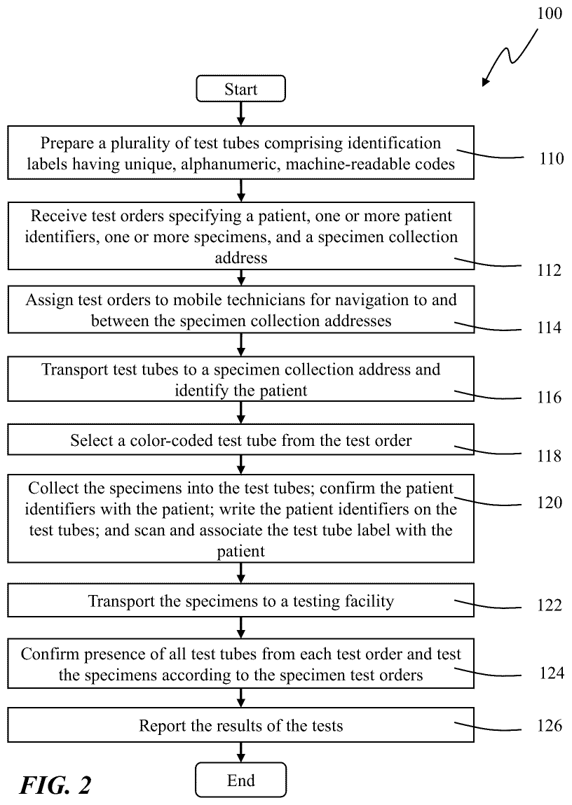

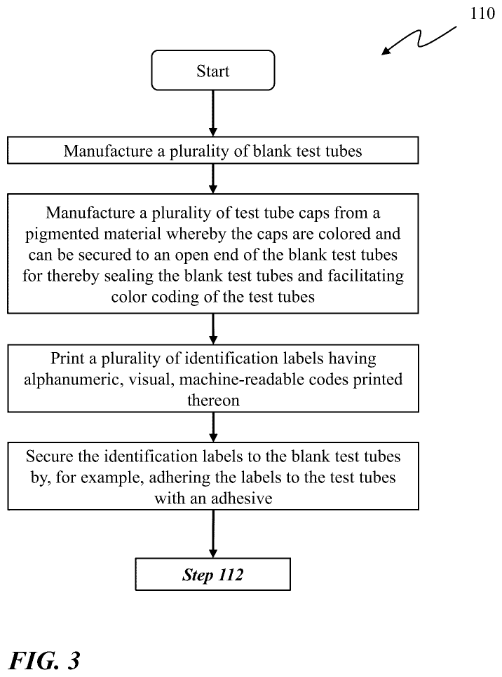

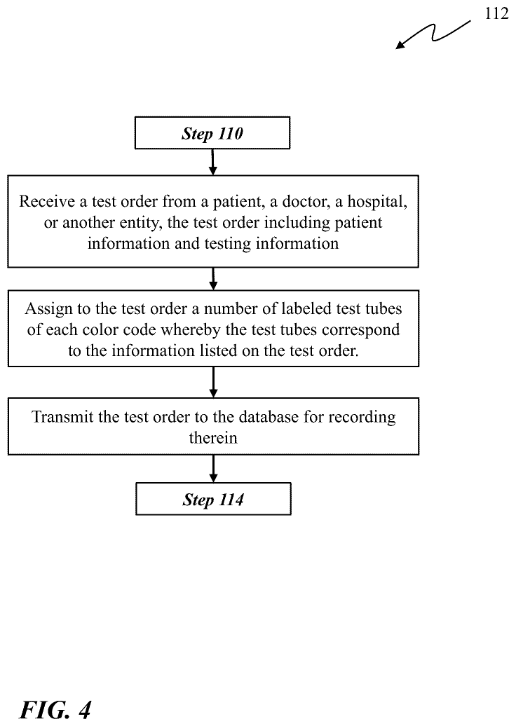

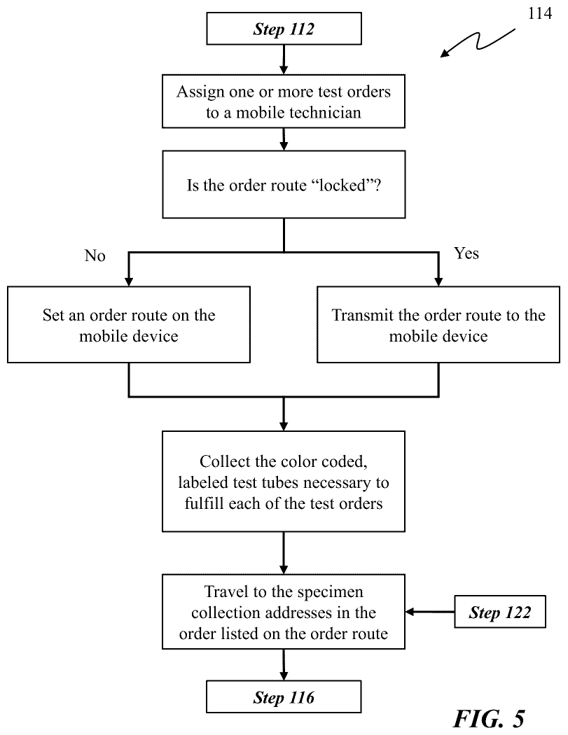

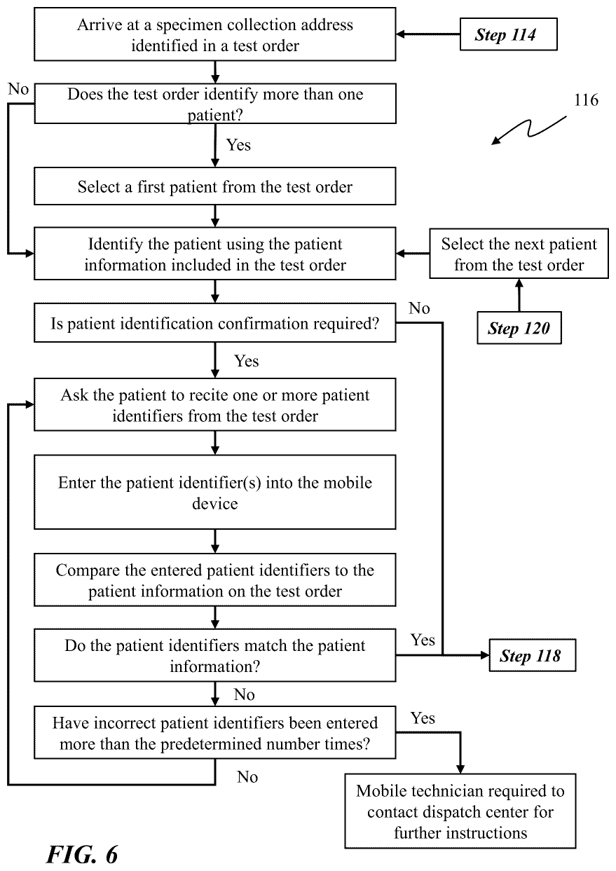

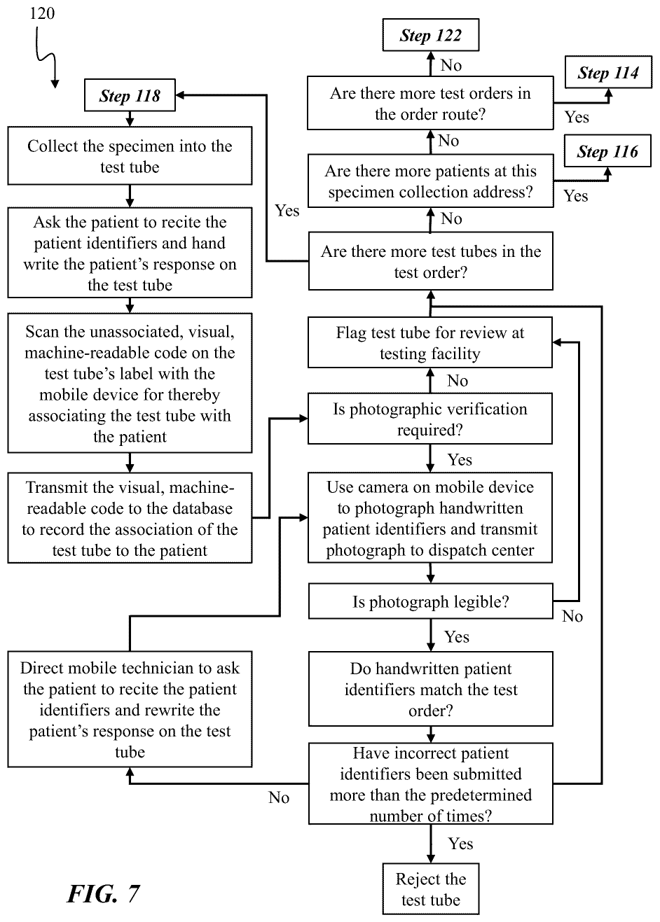

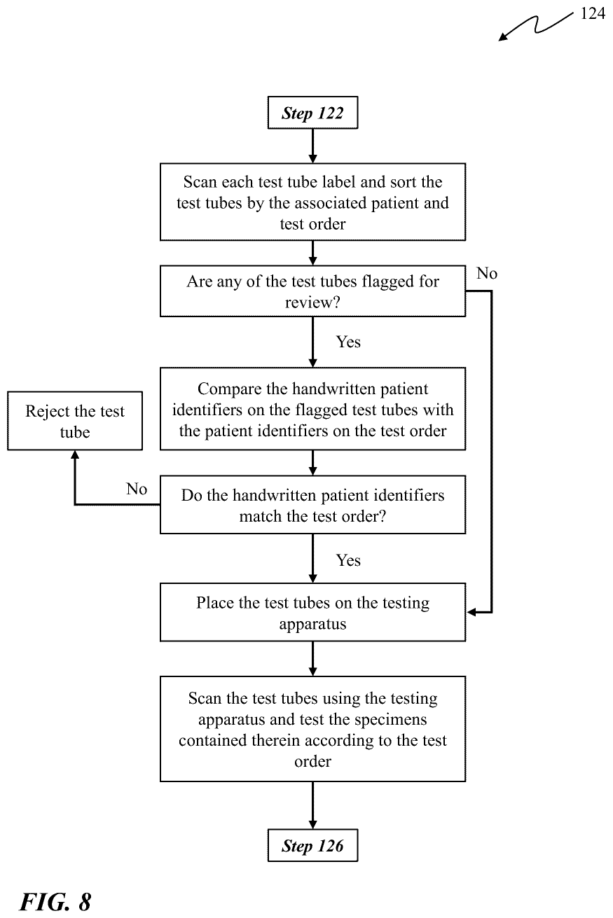

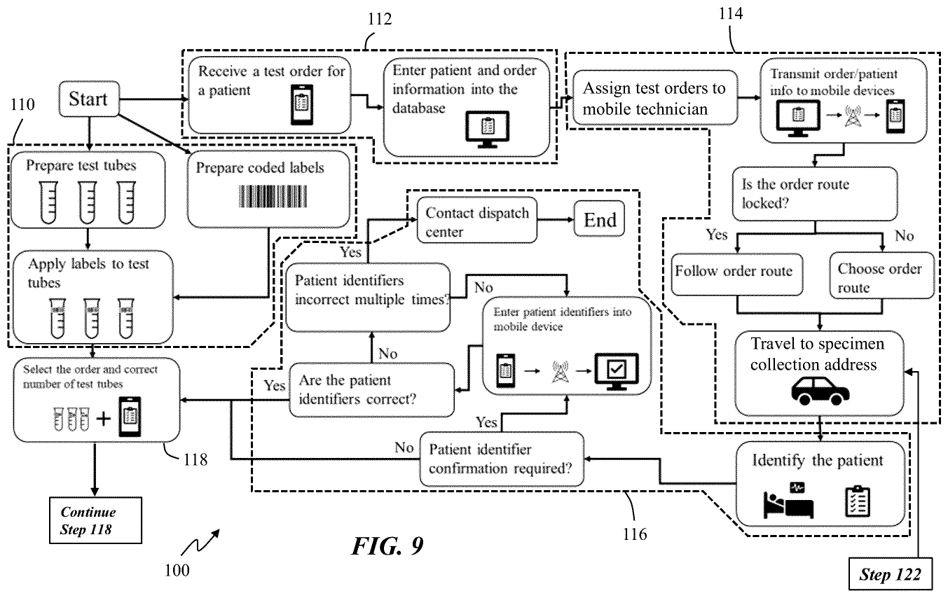

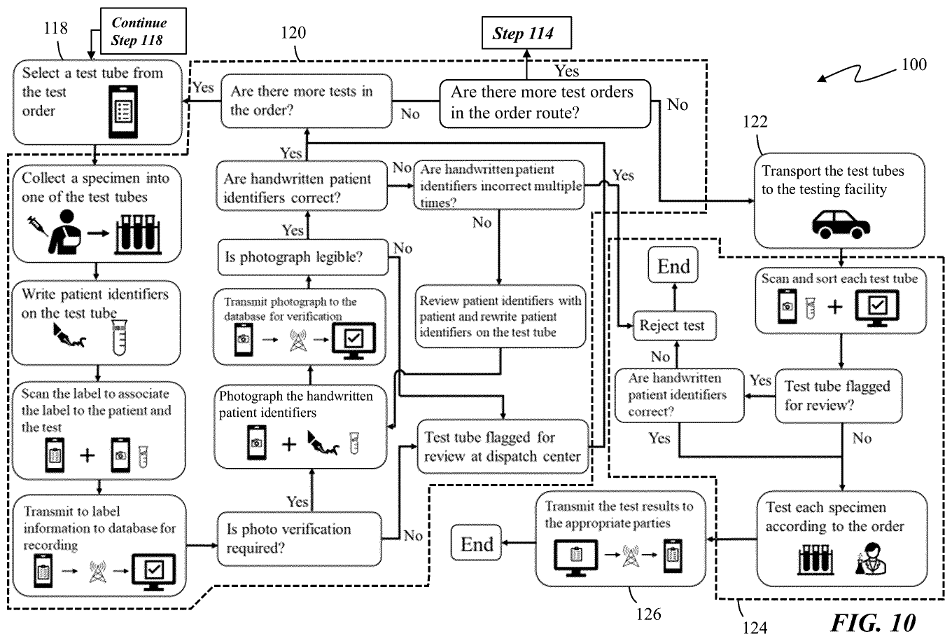

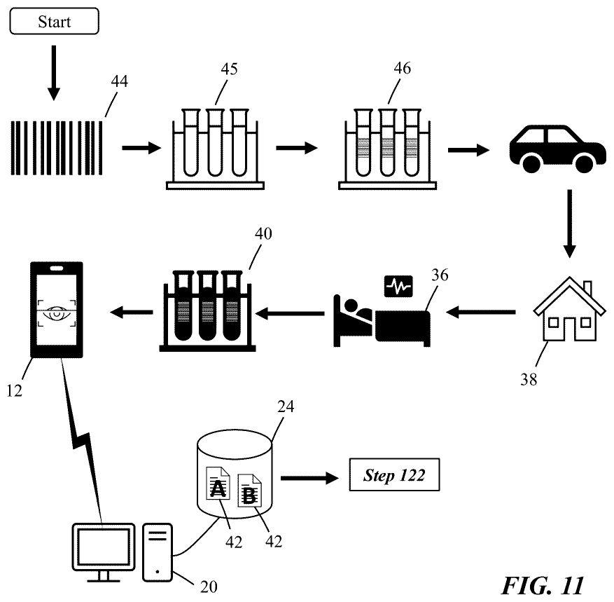

FIGS. 2-11 illustrate a method 100 for tracking and managing collection and transportation of specimens 40 by mobile technicians in accordance with the principles of the present invention. The specimens 40 can be collected into specimen containers such as, for example, labeled test tubes 46 comprising identification labels 44 and blank test tubes 45. Blank test tubes 45 can be manufactured using known and customary methods. Identification labels 44 can be printed using a label printer. Each identification label 44 can include an alphanumeric, visual, machine-readable code. The machine-readable codes are unique and are not initially associated with a patient 36. The machine-readable codes can be, for example, barcodes, QR codes, or any other type of visual, machine-readable code. The identification labels 44 can be applied and secured to the blank test tubes 45 using, for example, an adhesive.

The labeled test tubes 46 can include test tube caps manufactured in a known and customary manner. The test tube caps can be manufactured from pigmented materials, such as, for example, pigmented plastic for thereby facilitating color coding of the labeled test tubes 46. Preferably, chemicals/reagents may be added to the labeled test tubes 46 for a variety of purposes including, for example, facilitating preservation or testing of the specimens 40. Yet more preferably, the pigmented test tube caps can be configured for thereby color coding the labeled test tubes 46 according to the chemicals/reagents provided within each labeled test tubes 46.

Preferably, the visual, machine-readable codes on the identification labels 44 can be initially associated with the color code of the labeled test tube 46 and the expiration date of the labeled test tube 46. Accordingly, if, for example, a mobile technician selects and scans an expired labeled test tube 46 with their mobile device 12, the specimen ordering/tracking program 34 can generate and display an alert on the mobile device 12 indicating that the labeled test tube 46 has expired and directing the mobile technician to select a different labeled test tube 46. Additionally, if, for example, a mobile technician selects and scans a labeled test tube 46 which is not color coded for the particular specimen 40 specified on the test order 42, the specimen ordering/tracking program 34 can generate and display an alert on the mobile device 12 indicating that the mobile technician must select a different labeled test tube 46 having the correct color coding.

A test order 42 for a patient 36 can be created and transmitted, via the network 22, and recorded on the database 24. Preferably, test orders 42 are created on an ordering device 14, using the specimen ordering/tracking program 34, by a patient 36, a doctor, a hospital, or another entity, and transmitted, via the network 22, to a dispatch center device 18. The test orders 42 can then be reviewed, transmitted to the test order servers 20, via the network 22, and recorded on the database 24.

Each test order 42 can include patient information and test information. The patient information can include the patient's name, date of birth, address, and such other information as may be used to identify the patient 36 (the “patient identifiers”). The test information can include the remote patient specimen collection address 38, a list of the specimens 40 to be collected, and a list of the specimen tests to be performed on the specimens 40. Based on the number of specimens 40 and the types of tests ordered, a list of the corresponding color-coded labeled test tubes 46 can be created and recorded on the database 24.

Preferably, test orders 42 can also be created wherein only the remote patient specimen collection address 38 is specified. In this case, when the mobile technician arrives at the remote patient specimen collection address 38, the mobile technician can receive onsite orders for specimens 40 from patients 36, doctors, hospitals, and/or other entities present at the remote patient specimen collection address 38. In addition to patient information and test information, these onsite orders can also include additional information regarding the party requesting the specimens and tests and/or other information as desired or necessary. The onsite orders can then be recorded on the test order 42 by the mobile technician using the mobile devices 12.

Preferably, a group of test orders 42 can be assigned to a mobile technician by, for example, the dispatch personnel. Once assigned, the group of test orders 42 can be transmitted, via the network 22, to the mobile technician's mobile device 12. The labeled test tubes 46 required for each test order 42 can be collected from the dispatch center, the testing facility, or other test tube storage locations.

Preferably, for each group of test orders 42, the order in which the mobile technician collects the specimens 40, and the route by which the mobile technician travels to and between the remote patient specimen collection addresses 38 (the “order route”), can either be “locked” or “unlocked.” If an order route has been predetermined, the order route is considered “locked,” and the mobile technician can be restricted from changing the order route. If an order route has not been predetermined, the order route is “unlocked,” and the mobile technician may enter their own order route into their mobile device 12 as necessary or desired. As the specimens 40 are collected, the mobile technician can travel between the remote patient specimen collection addresses 38 in the order specified by the order route.

Preferably, as the mobile technician travels to and between the remote patient specimen collection addresses 38, the location tracking devices 30 on the mobile devices 12 can be configured to calculate and record the route and distance traveled by the mobile technician, for example, by tracking the longitude and latitude of the mobile devices 12. The order routes can be set and later adjusted for optimizing the order route as desired such as, for example, to minimize the distance traveled or to minimize time spent traveling between locations. The location information recorded by the location tracking devices 30 can be used for calculating the travel reimbursement amounts paid to the mobile technicians. The location information can also be used for calculating travel reimbursement amounts paid by the patient 36, doctor, hospital, health plan, insurance provider, or another entity, for the distance traveled to collect the specimens 40.

For a first test order 42, the mobile technician can transport one or more labeled test tubes 46 to the specified remote patient specimen collection address 38. When the mobile technician arrives at a remote patient specimen collection address 38, the mobile technician can identify a patient 36 using the patient information present in the test order 42. If the corresponding test order 42 identifies multiple patients 36, or multiple test orders 42 have specified the same remote patient specimen collection address 38, the mobile technician can identify each patient 36 using the corresponding patient information present in the test order(s) 42. Preferably, the mobile technician will select and identify a first patient 36, collect the corresponding specimens 40, and then repeat the process for each patient 36 present at the remote patient specimen collection address 38.

Preferably, after the mobile technician has identified a patient 36, the mobile technician will be required to perform a patient identifier confirmation. If a patient identifier confirmation is required, the mobile technician will ask the patient 36 to recite one or more of the patient identifiers listed in the test order 42. The patient identifiers can include, for example, the patient's name, date of birth, social security number, etc. The mobile technician can then enter the recited patient identifier into the mobile device 12, and the specimen ordering/tracking program 34, via the mobile device's processor, will compare the recited patient identifier entered by the mobile technician with the patient identifiers listed on the test order 42.

Preferably, if the recited patient identifiers do not match the test order 42, the mobile technician will be required to ask the patient 36 to recite the patient identifiers again, and the mobile technician can again enter the recited patient identifiers into the mobile device 12 for comparison against the test order 42. Yet more preferably, for each test order 42, a predetermined number of attempts to submit correct patient identifiers can be identified. If the mobile technician fails to submit the correct patient identifier within the predetermined number of attempts, the mobile technician can be required to contact the dispatch personnel for further instructions.

For example, after the mobile technician has identified a patient 36, the specimen ordering/tracking program 34 on the mobile technician's mobile device 12 can display text such as, “Please input Patient A's Date of Birth,” followed by an input box where the mobile technician can enter the date of birth recited by the patient 36. Using the mobile device's processor, the specimen ordering/tracking program 34 can then compare the date of birth entered by the mobile technician against the date of birth listed on the patient's test order 42. If the date of birth matches the test order 42, the specimen ordering/tracking program 34 can display text on the mobile technician's mobile device 12 confirming that the patient identifier confirmation has been successfully completed. Optionally, the predetermined number of attempts can be set by the dispatch personnel based on various factors including, for example, the mobile technician's historical performance record.

After a patient 36 has been identified, the mobile technician can select the corresponding test order 42 and can select an individual labeled test tube 46 specified by the test order 42. The mobile technician can then collect the corresponding specimen 40 into the labeled test tube 46 in a known and customary manner. Concurrently with collecting the specimen 40, the mobile technician can scan the machine-readable code on the test tube's identification label 44 using the mobile device's code sensor 26. The mobile device 12 can then transmit the machine-readable code to the test order servers 20. Preferably, the machine-readable code can be recorded as part of the corresponding test order 42 for thereby electronically associating the machine-readable code and the specimen 40 with the patient 36.

Preferably, and as may be specified by the test order 42 or required by government regulations, after collecting the specimen 40, the mobile technician can be required to ask the patient 36 to recite their patient identifiers, and the mobile technician can be required to write the recited patient identifiers on the test tube's identification label 44. The handwritten patient identifiers can then be compared with the patient identifiers specified on the corresponding test order 42 for confirming the identity of the patient 36 and the specimen 40.

Yet more preferably, a test order 42 can specify that a photograph verification of the patient identifiers written on the labeled test tube 46 is required. If a photograph verification is not required, a note can be recorded on the database 24 flagging the labeled test tube 46 for further review of the handwritten patient identifiers at the testing facility. If a photograph verification is required, the mobile technician can be prompted to use the mobile device's camera 28 to take a photograph of the labeled test tube 46. The photograph should show a clear image of the identification label 44 including the handwritten patient identifiers. The mobile device 12 can then transmit the photograph, via the network 22, to the dispatch center devices 18. If the photograph is illegible, a note can be recorded on the database 24 flagging the labeled test tube 46 for further review at the testing facility. If the photograph is legible, the dispatch personnel can compare the patient identifiers against the corresponding patient information recorded on the database 24. If the patient identifiers in the photograph are correct, the associated labeled test tube 46 is placed in a container and the mobile technician may proceed to the next test in the test order 42.

Preferably, the photograph can also include a clear image of the contents of the labeled test tube 46 for thereby allowing the photograph to be used for confirming a sufficient specimen 40 has been collected from the patient 36 to properly perform the ordered tests. For example, the photograph of the handwritten patient identifiers may further capture an image of the contents of the labeled test tube 46 whereby, when the photograph is transmitted for review, the reviewing personnel may further assess the sufficiency of the specimen 40 collected in the labeled test tube 46.

If the patient identifiers in the photograph are incorrect, the mobile technician can be prompted to review the patient identifiers with the patient 36. The mobile technician can then be required to rewrite the patient identifiers provided by the patient 36 on the identification label 44 and take a new photograph for verification by the dispatch personnel. For each test order 42, a predetermined number of attempts to submit correct patient identifiers can be identified. Optionally, the predetermined number of attempts can be set by the dispatch personnel based on various factors including, for example, the mobile technician's historical performance record. If the mobile technician fails to submit a photograph of correct, handwritten patient identifiers within the predetermined number of attempts, the labeled test tube 46 can be rejected and a new test order 42 can be required, or a note can be recorded on the test order 42 marking the labeled test tube 46 for additional review at the specimen testing facility.

Preferably, the mobile devices 12 are configured whereby the mobile technician can simultaneously scan the identification labels 44 with the code sensors 26 and photograph the identification labels 44 with the cameras 28. The machine-readable code can be transmitted by the mobile device 12 to the test order servers 20 for thereby recording the machine-readable code on the database 24, and, simultaneously, the photograph of the identification label 44 can be transmitted by the mobile device 12 to the dispatch center devices 18 for the photograph verification step.

The mobile technician can repeat the process described hereinabove for each of the test order 42 and specimens 40 assigned to the mobile technician.

After collecting any one or more specimens 40 specified by the assigned test orders 42, the mobile technician or another courier can transport the specimens 40 to a testing facility. At the testing facility, the identification labels 44 on each of the associated labeled test tubes 46 can be scanned using testing instrument's code sensor 26, and the labeled test tubes 46 can then be sorted according to the test orders 42.

Preferably, if an associated labeled test tube 46 has been flagged for further review, the handwritten patient identifiers on the identification label 44 can be compared with the patient identifiers listed on the test order 42. If the written identification information is incorrect, the associated labeled test tube 46 can be rejected and a new test order 42 can be required.

If a labeled test tube 46 has not been flagged for further review, or if the written patient identifiers have been verified as accurate, the labeled test tube 46 can be placed on a diagnostic machine 32. The diagnostic machine 32 can scan the identification label 44 to identify the particular test ordered for the specimen 40 and, thereafter, perform the diagnostic test ordered for the specimen 40. The results of such testing can then be transmitted to the appropriate parties via the network 22.

While this invention has been described as having an exemplary embodiment, the present invention may be further modified within the spirit and scope of this disclosure. This application is therefore intended to cover any variations, uses, or adaptations of the invention using its general principles.

What is claimed is:

1. A method for tracking specimens comprising the steps of:
at a first location, preparing a specimen container comprising an identification label having a unique, machine-readable code;
receiving a test order specifying one or more specimens to be collected from a patient at a remote patient specimen collection location;
recording the test order on an electronic test order database;
transporting the specimen container to the remote patient specimen collection location;
collecting the one or more specimens into the specimen container;
scanning the identification label unique, machine-readable code; and,
recording the unique, machine-readable code on the test order database;
wherein the unique, machine-readable code is recorded on the electronic test order database for electronically associating the specimen container with the patient.

2. The method of claim 1, wherein the specimen container includes a cap member having a color code.

3. The method of claim 1, wherein the unique, machine-readable code is an alphanumeric code.

4. The method of claim 2, wherein the unique, machine-readable code is an alphanumeric code, and wherein the alphanumeric unique, machine-readable code includes a first code portion that is electronically associated with the color code of the cap member.

5. The method of claim 1, wherein the test order specifies one or more patient identifiers.

6. The method of claim 5, further comprising the step of identifying the patient by directing the patient to recite from memory a recited patient identifier and comparing the recited patient identifier with the one or more test order patient identifiers.

7. The method of claim 1, wherein the unique, machine-readable code is a visual, machine-readable code.

8. The method of claim 7, wherein the unique, machine-readable code is a barcode or a QR code.

9. The method of claim 1, wherein the specimen container includes a cap member having a color code, wherein the unique, machine-readable code is an alphanumeric barcode, and wherein the alphanumeric barcode includes a first code portion that is electronically associated with the color code of the cap members.

10. The method of claim 9, wherein the test order further specifies a first color code.

11. The method of claim 10 further comprising the step of comparing the first code portion of the alphanumeric barcode with the first color code specified on the test order, wherein, if the first code portion is not electronically associated with the first color code, the specimen is rejected.

12. The method of claim 1 further comprising the step of transporting the specimen container to a third location and performing one or more tests on the one or more specimens.

13. A method for collecting and storing in an electronic database information about medical specimens, the method comprising the steps of:
at a first location, preparing a specimen container comprising an identification label having a unique, machine-readable code;
receiving a test order specifying a patient, one or more patient identifiers, one or more specimens to be collected, and a remote patient specimen collection location;
recording the test order on an electronic database;
physically transporting the specimen container to the remote patient specimen collection location;
collecting the one or more specimens into the specimen container;
scanning the identification label's machine-readable code; and,
transmitting the machine-readable code to be recorded on the electronic database;
wherein the machine-readable code identification label is recorded as part of the test order for electronically associating the specimen container with the patient.

14. The method of claim 13, wherein the step of scanning the identification label's machine-readable code and transmitting the machine-readable code to the electronic database is performed using a mobile device comprising a code scanning device, an input device, a display device, and a processor, wherein the code scanning device, the input device, and the display device are in communication with the processor, and wherein the mobile device is in communication with the electronic database.

15. The method of claim 14 further comprising the step of identifying the patient by directing the patient to recite from memory one or more recited patient identifiers, recording the one or more recited patient identifiers to the electronic database using the mobile device, and comparing the one or more recited patient identifiers with the one or more test order patient identifiers.

16. The method of claim 13, wherein the specimen container further comprises a color coded cap member, wherein the color code of the cap member is electronically associated with the machine-readable code, and wherein the color code indicates additional information regarding the specimen contained in the specimen container.

17. The method of claim 13 further comprising the step of physically transporting the specimen container to a testing location.

18. The method of claim 17 further comprising the step of performing one or more tests on the one or more specimens and recording one or more results of the one or more tests on the electronic test order database.
